(19) United States
(12) Patent Application Publication
(10) Pub. No.: US 2025/0282765 A1
(43) Pub. Date: Sep. 11, 2025

(54) RENEWABLE BIO-BASED, NON-TOXIC AROMATIC-FURANIC MONOMERS FOR USE IN THERMOSETTING AND THERMOPLASTIC POLYMERS

(71) Applicants: Drexel University, Philadelphia, PA (US); The Government of the United States, as represented by the Secretary of the Army, Washington, DC (US); Rowan University, Mullica Hill, NJ (US)

(72) Inventors: Craig Michael PAQUETTE, Ambler, PA (US); John Joseph LA SCALA, Wilmington, DE (US); Joshua Matthew SADLER, Parkville, MD (US); Giuseppe Rafaello PALMESE, Hainesport, NJ (US); Alexander William BASSETT, Mullica Hill, NJ (US); Joseph Francis STANZIONE, Wilmington, DE (US)

(73) Assignees: Drexel University, Philadelphia, PA (US); The Government of the United States, as represented by the Secretary of the Army, Washington, DC (US); Rowan University, Mullica Hill, NJ (US)

(21) Appl. No.: 19/217,667

(22) Filed: May 23, 2025

Related U.S. Application Data
(62) Division of application No. 17/284,032, filed on Apr. 9, 2021, now abandoned.
(60) Provisional application No. 62/744,198, filed on Oct. 11, 2018.

(51) Int. Cl. C07D 407/14; C07D 307/42; C08F 222/20; C08G 59/26

(52) U.S. Cl. CPC C07D 407/14; C07D 307/42; C08F 222/20; C08G 59/26

(57) ABSTRACT

A composition of matter including two optionally substituted phenol or optionally substituted aniline units separated by a furan spacer has been prepared. In particular, the chemical structure has a novolac bridge between the central furan ring and the two attached optionally substituted phenol and/or optionally substituted aniline units. These compounds can be modified to be used in various polymer resins. This new structure reduces toxicity relative to BPA/F, makes use of renewable chemicals, and produces certain beneficial polymer properties.

### CROSS REFERENCE TO RELATED APPLICATIONS

This application is a divisional of U.S. patent application Ser. No. 17/284,032, filed on Apr. 9, 2021, currently pending, which, in turn, is a 371 continuation of International patent application PCT/US19/55642, filed on Oct. 10, 2019, which, in turn, claims the benefit of U.S. Provisional Application No. 62/744,198, filed on Oct. 11, 2018, the entire disclosures of which is hereby incorporated by reference in their entirety as if set forth fully herein.

### STATEMENT OF GOVERNMENT INTEREST

This invention was made with government support under Contract Number DE-SC0014664 (Agreement No. 1120-1120-99) awarded by the Department of Energy, and Contract Numbers W911NF-15-2-0017, W911NF-16-2-0225, and W911NF-14-2-0086, awarded by the United States Army Research Laboratory. The Government has certain rights in the invention.

### FIELD OF THE INVENTION

The present invention relates to novel furan based amine and phenolic compounds with improved water barrier properties and reduced toxicity.

### BRIEF DESCRIPTION OF THE STATE OF THE ART

Bisphenol A (BPA) is produced by the coupling of phenol with acetone in the presence of an acid catalyst. The high isomeric purity, the ease of production, and the rigid aromatic structure of BPA made it a good candidate for incorporation into polymeric materials.

The production of BPA globally was estimated at around 12 billion pounds in 2011, and growing at 5% annually. While polycarbonate (74% of BPA use) and epoxy resin (20% of BPA use) applications comprised the vast majority of BPA utilization throughout the next several decades, BPA is also commonly used in applications such as thermal paper coatings, flame retardants, powder paints, and dye developers. Bisphenols such as bisphenol A (4,4′-isopropylidenephenol) have been used extensively in plastics and composites due to its aromaticity that provides high mechanical strength to BPA derived polymers. Industrially, these polymers are used in the manufacturing of goods such as metal food and beverage cans, epoxy resin linings, polycarbonate containers, helmets, headlight casings, composite resins, industrial/corrosion control coatings, and adhesives applications. BPA mimics estradiol, a hormone related to the development of reproductive tissue in several organisms including humans. Various effects at all stages of human development from fetal and neonatal growth to adult maturation have been linked to BPA exposure.

Since the realization of the hazardous effects of BPA exposure, chemists have sought to replace it with similarly high-performing, yet safer alternatives. However, this endeavor has proven difficult. Subtle alterations to the chemical structure of BPA often significantly decrease the properties imparted to the end polymer, or fail to sufficiently reduce its toxicity. Analogues such as bisphenol F (BPF), sulfur-bridged bisphenol (SBBP), oxygen-bridged bisphenol (OBBP), bisphenol S (BPS), bisphenol B (BPB), bisphenol E (BPE), and 4-cumylphenol (HHP) have been proven to be just as hazardous as BPA.

Industrial bisphenols are derived from petroleum, a non-renewable resource. Utilizing renewable sources of aromaticity, such as lignin, the second most abundant natural polymer rich in aromatic content, offers the potential to be a low cost sustainable alternative to petroleum feedstocks. On average, 70 million tons of lignin is produced as a waste product of the paper and pulping industry. The breakdown of lignin into monophenolics through processes such as pyrolysis is promising for the production of functionalized phenols that can be used as is or processed into specialty chemicals.

Bisguaiacol F (BGF) resembles BPF, except that it has methoxy groups pendant to the aromatic unit thereby significantly reducing toxicity. Recent research suggests that increasing the length of the unit that couples the two phenol units can also decrease toxicity effects. The phenyl-furan-phenyl derivatives (PFP) use both aspects of these technologies, with use of substituent groups on the aromatic unit and the use of a methylene-furan-methylene spacer. The combination should thus reduce toxicity even further. However, this BGF technology requires the use of vanillyl alcohol, which competes with food applications

Successful bisphenol alternatives must provide comparable or improved thermomechanical and optical properties, function as a drop in replacement, and have decreased toxicity and endocrine disruption potential. Many current alternatives provide similar properties but are difficult to synthesize and require expensive processing steps. These expensive synthesis steps limit their application as industrial alternatives to bisphenols. Other alternatives are derived from natural resources; however, typically these resources cannot sustain the production quotas necessary for industrial production. Furthermore, many other bisphenol alternatives are synthesized from toxic or volatile monomers such as formaldehyde and acetone.

Presently, there is a movement to remove BPA from baby food and beverage applications. However, BPA/F are still used extensively in many other applications, including food applications because of the low cost, large volumes, and the entrenchment of BPA/F into the chemical industry. The PFP technology has the potential to reduce toxicity and enable a similar but less toxic technology to be used for existing and new applications.

Industrial coatings, composites, and adhesives materials will likely be unaffected in the near future despite the toxicity of BPA. However, all food applications of BPA are likely to diminish rapidly regardless of government regulation because of public pressure. In the longer-term future, all applications of BPA will likely begin to be replaced with less toxic components.

### SUMMARY OF THE INVENTION

The present invention relates to furan compounds, epoxy thermosets made from the furan compounds as curing agents, polymers comprising the epoxy thermoset therein, and methods of preparing each of the foregoing.

The following sentences describe some embodiments of the invention.
1. In a first aspect, the disclosure relates to a furan containing compound according to Formula (I),

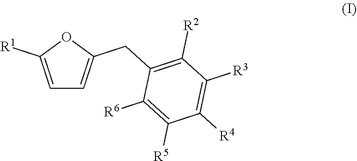

wherein R¹ is selected from H, and

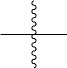

wherein

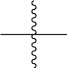

indicates a bond that is a point of attachment to a group according to Formula (II):

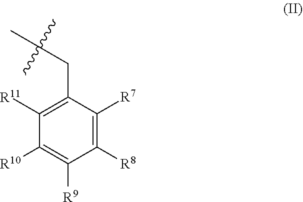

wherein R², R³, R⁴, R⁵, R⁶, R⁷, R⁸, R⁹, R¹⁰, and R¹¹ are each independently selected from: hydrogen, halogen, hydroxy, amino, nitro, cyano, carboxy, alkylamine residues having 1 to 18 carbon atoms, aminoalkyl residues having 1 to 18 carbon atoms, alkenylamine residues having 1 to 18 carbon atoms, aminoalkenyl residues having 1 to 18 carbon atoms, alkylamide residues having 1 to 18 carbon atoms, amidoalkyl residues having 1 to 18 carbon atoms, alkenylamide residues having 1 to 18 carbon atoms, amidoalkenyl residues having 1 to 18 carbon atoms, an optionally substituted alkyl group having 1 to 20 carbon atoms, an optionally substituted alkenyl group having 2 to 20 carbon atoms, an optionally substituted alkoxy group having 1 to 20 carbon atoms, an optionally substituted cycloalkyl group having 3 to 12 carbon atoms, an optionally substituted aryl group having 6 to 16 carbon atoms, and an optionally substituted heterocyclic group having 3 to 16 carbon atoms; wherein the alkyl group, the alkenyl group, the alkoxy group, the cycloalkyl group, the aryl group and the heterocyclic group can be substituted with 1 to 5 substituents independently selected from halogen, hydroxy, amino, nitro, cyano, carboxy, an alkyl group having 1 to 20 carbons, a heterocyclic group having 3 to 16 carbons, and an alkoxy group having 1 to 20 carbon atoms; wherein one or more of R²-R⁶ is hydrogen and one or more of R²-R⁶ is a hydroxy or amino; and wherein one or more of R⁷-R¹¹ is a hydroxy or amino.
2. The compound of sentence 1, wherein R¹ may be

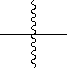

3. The compound of any one of sentences 1 or 2, wherein R², R³, R⁴, R⁵, R⁶, R⁷, R⁸, R⁹, R¹⁰, and R¹¹ may be each independently selected from hydrogen, hydroxy, alkenylamide residues having 1 to 18 carbon atoms, an alkyl group having 7 to 18 carbon atoms, an alkene group having 12 to 18 carbon atoms, an alkoxy group having 1 to 6 carbon atoms.
4. The compound of any one of sentences 1-3, wherein the furan containing compound may be prepared by reaction of 2,5-bishydroxymethyl furan or 2-hydroxymethyl furan and i) a phenolic compound selected from the group consisting of guaiacol, phenol, syringol, cardanol, cardol and capsaicin; or ii) an amino benzene selected from the group consisting of aniline, 2-anisidine, 3-anisidine, 4-anisidine, 2-toluidine, 3-toluidine, 4-toluidine, 2,5-dimethylaniline, 2,6-dimethylaniline, and 3,5-dimethylaniline.
5. The compound of any one of sentences 1-4, wherein the furan containing compound may be a compound of Formula (III):

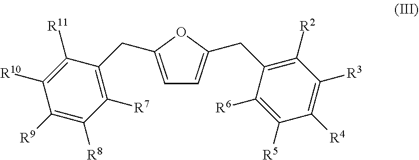

wherein R⁴ and R⁹ may be each independently selected from hydroxy or amino groups.
6. The compound of any one of the previous sentences, wherein R²-R⁶ may be hydrogen and two or three of R⁷-R¹¹ may be hydrogen; or preferably, three of R⁷-R¹¹ may be hydrogen.
7. The compound of any one of the previous sentences, wherein at least one of R²-R⁶ may be a hydroxy, and R⁴ may preferably be hydroxy; at least one of R⁷-R¹¹ may be a hydroxy, and R⁹ may preferably be hydroxy; at least one of R²-R⁶ may be an alkyl group having from 1 to 20 carbon atoms, preferably 5 to 17 carbon atoms, and even more preferably 15 carbon atoms; and at least one of R⁷-R¹¹ may be an alkyl group having from 1 to 20 carbon atoms, preferably 5 to 17 carbon atoms, and more preferably 15 carbon atoms.
8. The compound of sentence 1, wherein R¹ may be hydrogen.
9. The compound of sentence 8, wherein at least one of R²-R⁶ may be a hydroxy, preferably R⁴ may be hydroxy, and one of R²-R⁶ may be an alkyl group having from 1 to 20 carbon atoms, preferably 5 to 17 carbon atoms, and more preferably 15 carbon atoms.
10. In a second aspect, the present disclosure relates to a compound which is a reaction product prepared by the reaction of:
   i) the compound of Formula (I) wherein R¹ is

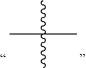

as recited in any one of claims 1 to 7; and ii) a reagent selected from one of the following:
a. a radically polymerizable monomer;
b. a halo-containing epoxide which is preferably epichlorohydrin;
c. at least one diacid, anhydride or diacyl chloride;
d. an isocyanate selected from hexamethylene diisocyanate, isophorone diisocyanate, and methylenediphenyl diisocyanate;
e. at least one compound selected from phosgene, diphosgene, triphosgene, and p-nitrophenyl chloroformate; and
f. a compound for converting a hydroxy to at least one of an amine and amide, wherein the compound is preferably 2-chloroacetamide and at least one of R²-R⁶ is a hydroxy and at least one of R⁷-R¹¹ is a hydroxy.
11. The compound of sentence 10, wherein the reaction product may be formed from the radically polymerizable monomer reagent, and the radically polymerizable monomer reagent may be selected from methacryloyl chloride, methacrylic anhydride, acryloyl chloride, acrylic anhydride, acrylic acid, and methacrylic acid, and wherein in the reaction product, a carbonyl of the radically polymerizable monomer is bonded to the oxygen from the hydroxy.
12. The compound of sentence 11, wherein the reaction may be catalyzed with a base catalyst, or the reaction is catalyzed with 4-(dimethylamino)pyridine or trimethylamine.
13. The compound of any one of sentences 10-12, wherein the reaction product may be formed from the radically polymerizable monomer reagent, the radically polymerizable monomer reagent is selected from methacryloyl chloride, methacrylic anhydride, methyl methacrylate, and methacrylic acid and the reaction product is a product of Formula (IV):

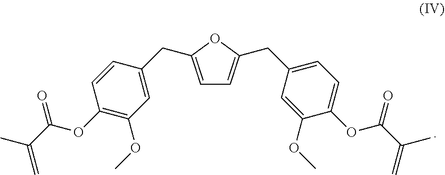 [SMILES: C=C(C)C(=O)Oc1ccc(Cc2ccc(Cc3ccc(OC(=O)C(=C)C)c(OC)c3)o2)cc1OC]

14. The compound of any one of sentences 10-12, wherein the reaction product may be formed from the radically polymerizable monomer reagent, the radically polymerizable monomer reagent is selected from acryloyl chloride and acrylic anhydride and the reaction product is a product of Formula (V):

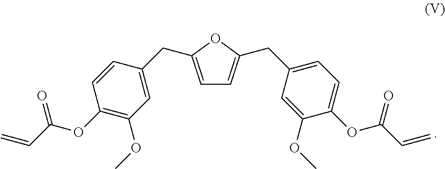 [SMILES: C=CC(=O)Oc1ccc(Cc2ccc(Cc3ccc(OC(=O)C=C)c(OC)c3)o2)cc1OC]

15. In a third aspect, the present disclosure relates to a polymer produced by radical polymerization of the reaction product of any one of sentences 11-14 formed by reaction with the radically polymerizable monomer reagent.
16. The polymer of sentence 15, wherein the radical polymerization may be initiated with a free radical initiator, which is preferably cumene hydroperoxide or methyl ethyl ketone peroxide.
17. The polymer of any one of sentences 15-16, wherein the radical polymerization may be performed with a promoter, which is preferably cobalt naphthenate or dimethyl aniline.
18. In a fourth aspect, the present disclosure relates to a polymer produced by further reacting the reaction product of any one of sentences 11-14 formed with the radically polymerizable monomer reagent, with a reactive diluent, which is preferably selected from styrene, methacrylated lauric acid, and furfuryl methacrylate.
19. The polymer of sentence 18, wherein 30-90 wt. % of the reaction product formed with the radically polymerizable monomer reagent may be reacted with 10-70 wt. % of the reactive diluent, or preferably 50-75 wt. % of the reaction product formed with the radically polymerizable monomer reagent is reacted with 25-50 wt. % of the reactive diluent.
20. The polymer of sentence 19 may have a Tg of 160-200° C., or preferably may have a Tg of about 186° C., as determined by DSC at 10° C./min, and may have a maximum degradation rate at temperature of 360-400° C., or preferably about 380° C., as determined by TGA in nitrogen at 10° C./min.
21. The compound of sentence 10, wherein the reaction product may be formed from the compound of Formula (I) and the halo-containing epoxide which is preferably epichlorohydrin.
22. The compound of sentence 21, wherein the reaction between the compound of Formula (I) and the halo-containing epoxide may be performed in the presence of a base, which is preferably sodium hydroxide or potassium hydroxide, and the sodium hydroxide or the potassium hydroxide is in aqueous solution and may have a pH of from 13 to 14.
23. The compound of any one of sentences 21-22, wherein the reaction between the compound of Formula (I) and the reagent which is the halo-containing epoxide may be catalyzed by a phase transfer catalyst, which is preferably a quaternary ammonium salt, or n-butyl ammonium bromide.
24. The compound of sentence 10, wherein at least one of R²-R⁶ may be a hydroxy and at least one of R⁷-R¹¹ may be a hydroxy, the reaction product is formed from the halo-containing epoxide, and is substituted on the oxygen of one said hydroxy group of R²-R⁶ and on the oxygen of one said hydroxy group of R⁷-R¹¹ with alkyl epoxy groups, and preferably, R⁴ and R⁹ are hydroxy.
25. The compound of sentence 24, wherein the reaction product may be:

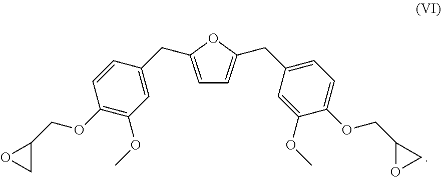 [SMILES: COc1cc(Cc2ccc(Cc3ccc(OCC4CO4)c(OC)c3)o2)ccc1OCC1CO1]

26. In a fifth aspect, the present disclosure relates to an epoxy thermoset formed by curing, in the presence of at least one epoxy curing agent, the reaction product of claim 10 formed from the compound of Formula (I) and the halo-containing epoxide.
27. The epoxy thermoset of sentence 26, wherein the epoxy curing agent may be an aliphatic polyamine, which is preferably diethylenetriamine (DETA), triethylenetetramine (TETA), tetraethylenepentamine (TEPA), diproprenediamine (DPDA), or dimethylaminopropylamine (DEAPA); or an alicyclic polyamine which is preferably N-aminoethylpiperazine (N-AEP), 4,4′-diaminodicyclohexylmethane (AMICURE® PACM), menthane diamine (MDA), or isophoronediamine (IPDA); or an aliphatic aromatic amine which is preferably m-xylenediamine (m-XDA); or an aromatic amine which is preferably metaphenylene diamine (MPDA), diaminodiphenylmethane (DDM), diaminodiphenylsulfone (DDS); or EPIKURE® Curing Agent W; or nadic methyl anhydride, phthalic anhydride dicyandiamide, nadic anhydride, and dicyandiamide, hexahydrophthalic anhydride (HHPA), methylhexahydrophthalic anhydride (MHHPA) and methyltetrahydrophthalic anhydride (MTHPA).
28. In a sixth aspect, the present disclosure relates to a polymerizable reaction product of the compound of claim 21 and a radically polymerizable monomer selected from acrylic acid and methacrylic acid,
29. The polymerizable reaction product of sentence 28, wherein a molar ratio of the radically polymerizable monomer to the compound of claim 21 may be from 1:1 to 2:1, preferably from 1.1:1 to 1.5:1.
30. The compound of any one of sentences 28 and 29, wherein the polymerizable reaction product may further comprise a reactive diluent, and said reactive diluent is preferably styrene, methacrylated lauric acid, or furfuryl methacrylate.
31. The compound of any one of sentences 28 and 29, wherein the radically polymerizable monomer may be acrylic acid and forms a reaction product according to Formula (VII):

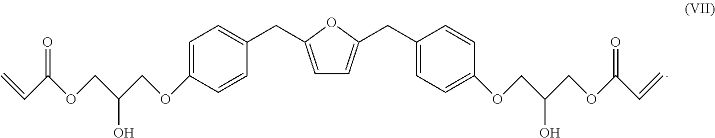 [SMILES: C=CC(=O)OCC(O)COc1ccc(Cc2ccc(Cc3ccc(OCC(O)COC(=O)C=C)cc3)o2)cc1]

32. The compound of any one of sentences 28 and 29, wherein the radically polymerizable monomer may be methacrylic acid and forms a reaction product according to Formula (VIII):

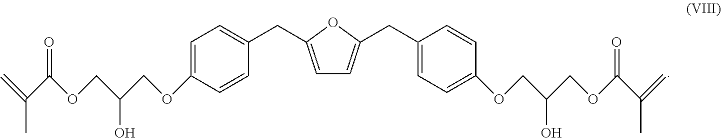 [SMILES: C=C(C)C(=O)OCC(O)COc1ccc(Cc2ccc(Cc3ccc(OCC(O)COC(=O)C(=C)C)cc3)o2)cc1]

33. The compound of sentence 10, wherein the reagent may be selected from at least one diacid, at least one anhydride, at least one diacyl chloride and mixtures thereof.
34. The compound of sentence 33, wherein the reagent may be selected from maleic anhydride, phthalic anhydride, terephthalic acid and adipic acid.
35. The compound of any one of sentences 33 and 34, wherein the reaction product may also be reacted with a diol or a polyol.
36. The compound of any one of sentences 33 or 34, wherein the reaction product may be according to Formula (IX):

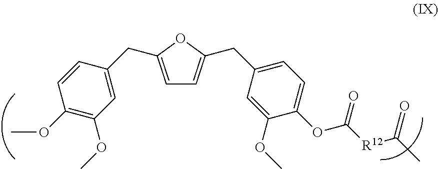

wherein R¹² is an optionally substituted alkylene group having 1 to 20 carbon atoms, an optionally substituted alkenylene group having 2 to 20 carbon atoms, an optionally substituted divalent heterocyclic group having 3 to 15 carbon atoms, an optionally substituted arylene group having 6 to 15 carbon atoms and an optionally substituted cycloalkylene group having 3 to 12 carbon atoms; and each group of R¹² is optionally substituted with 1 to 4 substituents independently selected from halogen, hydroxy, amino, nitro, cyano, carboxy, an alkyl group having 1 to 20 carbons, a heterocyclic group having 3 to 16 carbons, and an alkoxy group having 1 to 20 carbon atoms.
37. In a seventh aspect, the present disclosure relates to a curable reaction product obtainable by reacting a compound of any one of claims 33-36 with at least one olefinically unsaturated reactive diluent, which is preferably styrene, methacrylated lauric acid, or methyl methacrylate.
38. The curable reaction product of sentence 37, wherein 30-90 wt. % of the compound of any one of claims 33-36 may be reacted with 10-70 wt. % of the reactive diluent, or preferably 50-75 wt. % of the compound of any one of claims 33-36 is reacted with 25-50 wt. % of the reactive diluent.
39. In an eighth aspect, the present disclosure relates to a cured thermoset obtainable by curing the curable reaction product of any one of sentences 37 and 38 with a free radical initiator, which is preferably cumene hydroperoxide and methyl ethyl ketone peroxide.
40. The cured thermoset of sentence 39, wherein the curing may be performed in a presence of a promoter, which is preferably cobalt naphthenate or dimethyl aniline.
41. The compound of any one of sentences 33-36, wherein the reaction product may be formed from the compound of Formula (I) and the reagent which is the diacid, anhydride or diacyl chloride, and wherein a molar ratio of the compound of Formula (I) to the reagent is 1:0.8 to 0.8:1, or preferably the molar ratio is about 1:1.
42. The compound of sentence 10, wherein the reagent may be an isocyanate derivative, and wherein the isocyanate derivative is preferably selected from toluene diisocyanate, hexamethylene diisocyanate, methylene diphenyl diisocyanate, and isophorone diisocyanate.
43. The compound of sentence 20, wherein the reaction product may form a compound according to Formula (X):

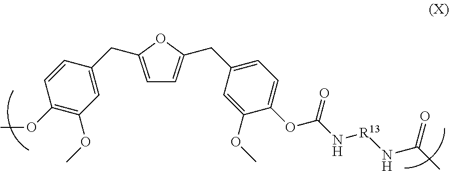

wherein R¹³ is an optionally substituted alkylene group having 1 to 20 carbon atoms, an optionally substituted alkenylene group having 2 to 20 carbon atoms, an optionally substituted divalent heterocyclic group with 3 to 15 carbon atoms, an optionally substituted arylene group having 6 to 15 carbon atoms and an optionally substituted cycloalkylene group having 3 to 12 carbon atoms; and R¹³ is optionally substituted with 1 to 4 substituents independently selected from halogen, hydroxy, amino, nitro, cyano, carboxy, an alkyl group having 1 to 20 carbons, a heterocyclic group having 3 to 16 carbons, and an alkoxy group having 1 to 20 carbon atoms.
44. The compound of sentence 10, wherein the reagent may be selected from phosgene, diphosgene and triphosgene and p-nitrophenyl chloroformate.
45. The compound of sentence 44, wherein the reaction product may form a compound according to Formula (XI):

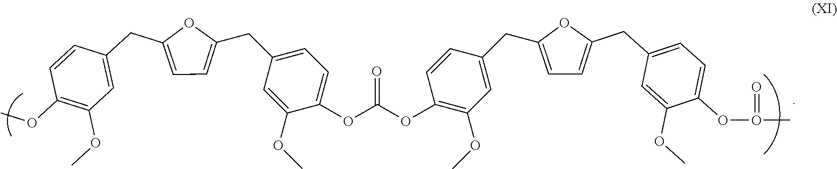

46. The compound of sentence 10, wherein the reagent may be 2-chloroacetamide.
47. The compound of sentence 46, wherein the reaction product may form a compound according to Formula (XII):

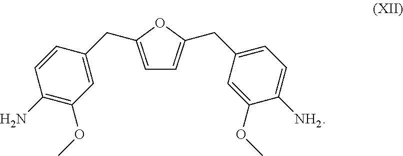 [SMILES: COc1cc(Cc2ccc(Cc3ccc(N)c(OC)c3)o2)ccc1N]

48. In a ninth aspect, the present disclosure relates to a compound of formula (XIII) obtainable by reaction of a compound of Formula (XII) with an isocyanate preferably selected from toluene diisocyanate, hexamethylene diisocyanate, methylene diphenyl diisocyanate, and isophorone diisocyanate, to form an isocyanate compound according to Formula (XIII):

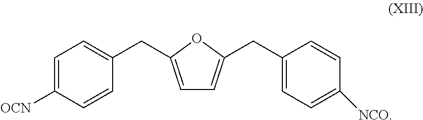

49. In a tenth aspect, the present disclosure relates to a method of preparing a compound of Formula (IV):

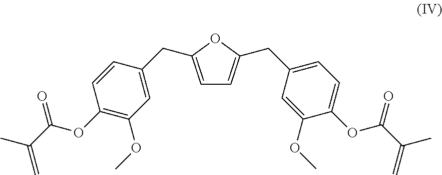 [SMILES: C=C(C)C(=O)Oc1ccc(Cc2ccc(Cc3ccc(OC(=O)C(=C)C)c(OC)c3)o2)cc1OC]

by reacting a compound of the Formula (III) of claim 5, wherein R², R³, R⁶, R⁷, R¹⁰, and R¹¹ are hydrogen, R⁴ and R⁹ are hydroxy, and R⁵ and R⁸ are methoxy, with a radically polymerizable monomer selected from methacryloyl chloride and methacrylic anhydride, in a presence of a base catalyst and an aprotic solvent, wherein the base catalyst may be selected from 4-(dimethylamino)pyridine and triethylamine and the aprotic solvent may be selected from dichloromethane and tetrahydrofuran, and at a temperature of from 20° C. to 80° C.
50. In an eleventh aspect, the present disclosure relates to a method of preparing the compound of Formula (V):

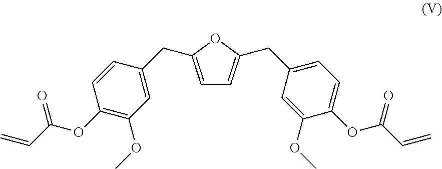 [SMILES: C=CC(=O)Oc1ccc(Cc2ccc(Cc3ccc(OC(=O)C=C)c(OC)c3)o2)cc1OC]

by reacting a compound of the Formula (III) of claim 5, wherein R², R³, R⁶, R⁷, R¹⁰, and R¹¹ are hydrogen, R⁴ and R⁹ are hydroxy, and R⁵ and R⁸ are methoxy, with a radically polymerizable monomer, selected from acryloyl chloride and acrylic anhydride, in a presence of a base catalyst and an aprotic solvent, wherein the base catalyst may be selected from 4-(dimethylamino)pyridine and triethylamine; and the aprotic solvent may be selected from dichloromethane and tetrahydrofuran, and at a temperature of from 20° C. to 80° C.
51. The method of sentence 49 or 50, wherein the temperature may be from 25° C. to 55° C.
52. In a twelfth aspect, the present disclosure relates to a method of preparing an epoxy derivative of Formula (VI):

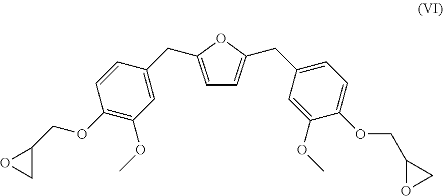 [SMILES: COc1cc(Cc2ccc(Cc3ccc(OCC4CO4)c(OC)c3)o2)ccc1OCC1CO1]

by reacting the compound of Formula (III) of claim 5, wherein R², R³, R⁶, R⁷, R¹⁰, and R¹¹ are hydrogen, R⁴ and R⁹ are hydroxy, and R⁵ and R⁸ are methoxy, with excess epichlorohydrin at a temperature of from 15° C. to 60° C. with a quaternary ammonium salt, followed by addition of an alkali base selected from sodium hydroxide and potassium hydroxide, at a temperature of 0° C. to 103° C. in water, followed by extraction of salts and distillation.
53. The method of sentence 52, wherein the compound of the Formula (III) may be present in a reaction mixture for the reaction in an amount of 10 to 11 mol %.
54. In a thirteenth aspect, the present disclosure relates to a method of producing the compound of sentence 25, wherein the compound of Formula (III) wherein R², R³, R⁶, R⁷, R¹⁰, and R¹¹ are hydrogen, R⁴ and R⁹ are hydroxy, and R⁵ and R⁸ are methoxy, is reacted with excess epichlorohydrin at a temperature of from 20° C. to 25° C. and an alkali base is added at a temperature of from 0° C. to 5° C.
55. In a fourteenth aspect, the present disclosure relates to a method of preparing a compound of Formula (VII):

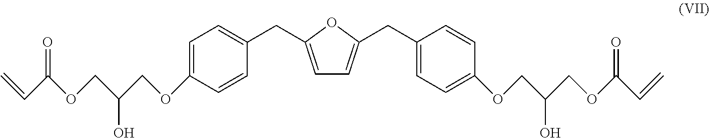 [SMILES: C=CC(=O)OCC(O)COc1ccc(Cc2ccc(Cc3ccc(OCC(O)COC(=O)C=C)cc3)o2)cc1]

comprising reacting the epoxy derivative of Formula (VI) of claim 25 with excess acrylic acid at a temperature of from 70° C. to 120° C., for 1 to 5 hours, in the presence of a catalyst.
56. In a fifteenth aspect, the present disclosure relates to a method of preparing a compound of Formula (VIII):

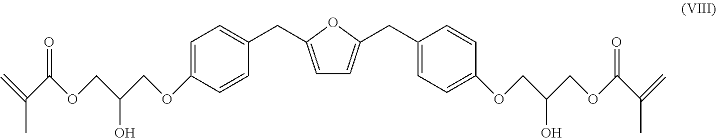 [SMILES: C=C(C)C(=O)OCC(O)COc1ccc(Cc2ccc(Cc3ccc(OCC(O)COC(=O)C(=C)C)cc3)o2)cc1]

comprising reacting the epoxy derivative of Formula (VI) of claim 25, with excess methacrylic acid at a temperature of from 70° C. to 120° C., for 1 to 5 hours, in the presence of a catalyst.
57. The method of any one of sentences 55 or 56, wherein the catalyst may be selected from a chromium (III)-based organometallic compound (AMC-2), triphenylphosphine, and triphenylantimony (III), imidizole.
58. The method of any one of sentences 55-57, wherein the temperature may be from 90° C. to 100° C.
59. The method of any one of sentences 55-58, wherein the reagents may be reacted for 2 to 3 hours.
60. In a sixteenth aspect, the present disclosure relates to a method of preparing the compound of Formula (IX):

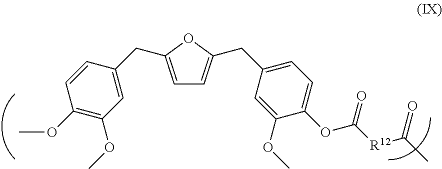

comprising melting the compound of Formula (III) of claim 5, wherein R², R³, R⁶, R⁷, R¹⁰, and R¹¹ are hydrogen, R⁴ and R⁹ are hydroxy, and R⁵ and R⁸ are methoxy, in the presence of a diacid, and a catalyst,
   wherein the diacid may be selected from maleic anhydride phthalic anhydride, terephthalic acid and adipic acid,
   wherein the catalyst may be selected from:
      p-toluenesulfonc acid, and a macro reticular polystyrene based ion exchange resin with a strongly acidic sulfonic group (AMBERLYST 15 or DOWEX DR-2030) and
   wherein the reaction is carried out at a temperature of from 55° C. to 220° C.

61. The method of sentence 60, wherein a reaction mixture used for the reaction may further comprise a diol or a polyol, and wherein the diol or polyol may be selected from diethylene glycol, isosorbide, and propylene glycol.

62. The method of any one of sentences 60 or 61, wherein the reaction may be carried out at a temperature of from 125° C. to 180° C.

63. The method of any one of sentences 60-62, wherein the reaction may be carried out in a presence of an azeotropic solvent, and wherein the solvent may be selected from toluene and xylene.

64. The method of any one of sentences 60-63, wherein the reaction may be carried out in the absence of an azeotropic solvent.

65. In a seventeenth aspect, the present disclosure relates to a method of preparing a compound of Formula (X):

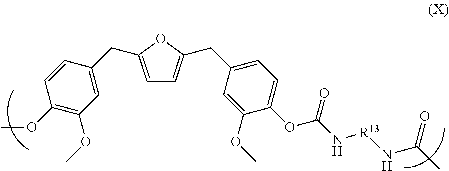

wherein R¹³ is an optionally substituted alkylene group having 1 to 20 carbon atoms, an optionally substituted alkenylene group having 2 to 20 carbon atoms, an optionally substituted divalent heterocyclic group with 3 to 15 carbon atoms, an optionally substituted arylene group having 6 to 15 carbon atoms and an optionally substituted cycloalkylene group having 3 to 12 carbon atoms; and R¹³ is optionally substituted with 1 to 4 substituents independently selected from halogen, hydroxy, amino, nitro, cyano, carboxy, an alkyl group having 1 to 20 carbons, a heterocyclic group having 3 to 16 carbons, and an alkoxy group having 1 to 20 carbon atoms;
   comprising a step of dissolving the compound of Formula (III) of claim 5, wherein R², R³, R⁶, R⁷, R¹⁰, and R¹¹ are hydrogen, R⁴ and R⁹ are hydroxy, and R⁵ and R⁸ are methoxy, in a solvent with an isocyanate derivative, followed by adding a catalyst, at a temperature of from 0° C. to 125° C., wherein the solvent may be selected from tetrahydrofuran, chloroform, and diethyl ether;
   wherein the isocyanate derivative may be selected from toluene diisocyanate, hexamethylene diisocyanate, methylene diphenyl diisocyanate, and isophorone diisocyanate; and
   wherein the catalyst may be selected from trimethylamine, pyridine, and 1,8-diazabicyclo[5.4.0]undec-7-ene.
66. The method of sentence 65, wherein in a reaction mixture used in the method, the catalyst may be present in an amount of 1 to 25 mol %, or from 5 to 15 mol %, based on a total of the moles in the reaction mixture.
67. The method of any one of sentences 65 or 66, wherein in a reaction mixture used in the method, the isocyanate derivative may be present in the reaction mixture in an amount of from 25 to 75 mol % and the compound of Formula (III) is present in the reaction mixture in an amount of from 25 to 75 mol %.

68. The method of any one of sentences 65-67, wherein in a reaction mixture used in the method the isocyanate derivative may be present in the reaction mixture in an amount of from 33 to 67 mol % and the compound of Formula (III) is present in the reaction mixture in an amount of from 33 to 67 mol %.
69. The method of any one of sentences 65-68, wherein the reaction may be carried out at a temperature of from 25° C. to 80° C.
70. In a eighteenth aspect, the present disclosure relates to a method of preparing the compound of Formula (XI):

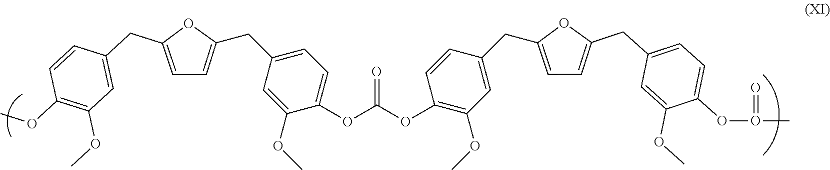

comprising a step of reacting the compound of Formula (III) of claim 5, wherein R², R³, R⁶, R⁷, R¹⁰, and R¹¹ are hydrogen, R⁴ and R⁹ are hydroxy, and R⁵ and R⁸ are methoxy, with a reagent selected from phosgene, diphosgene, triphosgene, and p-nitrophenyl chloroformate, in the presence of a catalyst at a temperature of from 0° C. to 100° C.
71. The method of sentence 70, wherein the reaction may be carried out in the presence of a solvent, wherein the solvent may be selected from 1,4-dioxane, acetonitrile, and dichloromethane.
72. The method of any one of sentences 70 or 71, wherein the catalyst may be selected from pyridine, 4-(dimethylamino)pyridine, 1-methylimidazole and 2-methylimidazole.
73. The method of any one of sentences 70-72, wherein the catalyst may be present in an amount of from 0.5 to 10 mol %, or from 1 to 5 mol %, based on the total moles in the reaction mixture.
74. The method of any one of sentences 70-73, wherein a second catalyst selected from trimethylamine and pyridine may be present during the reaction.
75. In a nineteenth aspect, the present disclosure relates to a method of preparing a compound of Formula (XII):

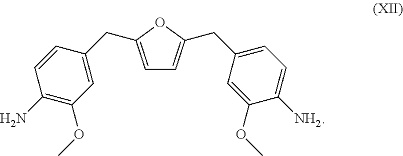 [SMILES: COc1cc(Cc2ccc(Cc3ccc(N)c(OC)c3)o2)ccc1N]

comprising reacting the compound of Formula (III) of claim 5, wherein R², R³, R⁶, R⁷, R¹⁰, and R¹¹ are hydrogen, R⁴ and R⁹ are hydroxy, and R⁵ and R⁸ are methoxy, with excess 2-chloroacetamide, in a presence of a catalyst, wherein the catalyst is selected from potassium carbonate, and potassium iodide, and a solvent, wherein the solvent is selected from dichloromethane, dimethylformamide, and chloroform, preferably selected from dichloromethane and chloroform, at a temperature of from 50° C. to 100° C. for 1 hour, followed by increasing the temperature to a range of 125° C. to 175° C. for 4 hours.
76. The compound or method of any one of the previous sentences, wherein
   the alkyl group is selected from a straight or branched chain methyl, ethyl, propyl, butyl, pentyl, hexyl, heptyl, octyl, nonyl, decyl, undecyl and dodecyl group,
   the alkene group is selected from a vinyl, propenyl, or a straight or branched chain butenyl, pentenyl, hexenyl, heptenyl, octenyl, nonenyl, decenyl, undecenyl and dodecenyl group,
   the alkoxy group is selected from a straight or branched chain methoxy, ethoxy, propoxy, butoxy, pentoxy, hexoxy, heptoxy, octoxy, nonoxy, decoxy, undecoxy and dodecoxy group
   the cycloalkyl group is selected from a cyclopentyl group and a cyclohexyl group,
   the aryl group is selected from a phenyl, a tolyl, and a biphenyl group,
      the heterocyclic group is selected from pyrrolidine, pyrrole, tetrahydrofuran, furan, tetrahydrothiophene, thiophene, imidazolidine, pyrazolidine, imidazole, pyrazole, oxazolidine, isoxazolidine, oxazole, isoxazole, thiazolidine, isothiazolidine, thiazole, isothiazole, dioxolane, dithiolane, piperidine, pyridine, bipyridine, tetrahydropyran, pyran, piperazine, diazines, morpholine, oxazine, thiomorpholine, and thiazine; and
   each of the foregoing groups are optionally substituted with 1-4 substituents and the optional substituents are selected from the group consisting of an alkyl group having 1 to 3 carbons, an aldehyde, a hydroxyl group and methoxy group.
77. The compound or method of any one of the previous sentences, wherein the portion of the structure within the parenthesis may be a repeat unit that repeats 2-10,000 times or 2-5,000 times or 2-1,000 times, or 2-500 times or 2-100 times, or 2-50 times.

### BRIEF DESCRIPTION OF THE DRAWINGS

FIG. 1 shows a reaction scheme for preparing phenyl-furan-phenyl using cardanol as the phenolic compound.

FIG. 2 shows a reaction scheme for preparing furans from furfuryl alcohol reacted with phenolic compounds.

### DETAILED DESCRIPTION OF THE PREFERRED EMBODIMENTS

The invention relates to the development of mixed furan phenols derived from feedstocks including but not limited to plant sugars and phenols. Starting chemicals such as guaiacol and bis-hydroxymethylfuran (bHMF) are reacted to form phenyl-furan-phenyl derivatives (PFP). Uses of these materials include but are not limited to the use as feedstocks into novel monomer units for polymers. Preparation of PFPs into monomers and polymers is not complex and thus economically viable.

bHMF is very reactive towards phenolic compounds and attaches readily at the site para to the phenolic hydroxyl group with high selectivity, although some reaction at the meta and ortho positions also occurs. Both furan methylene hydroxyl groups are reactive in this way. Furthermore, unlike furfural alcohol which contains a single furan methylene hydroxyl group, the bHMF methylene hydroxyl groups are not highly reactive with themselves because they strongly prefer to attach to the carbon next to the oxygen heteroatom in the furan ring, both of which positions are occupied in bHMF.

The reaction proceeds readily under acidic conditions using HCl, p-toluene sulfonic acid or solid catalysts such as Dowex. The reaction is run at moderate temperatures, ˜60° C., for a few hours until complete coupling occurs as verified by NMR.

Bis-guaiacol F (BGF) is less toxic than BPA and BPF because the phenolic methoxy groups limit the ability of the molecule to interact with the estrogenic receptor. Thus, a PFP using guaiacol as the starting phenolic is beneficial from this perspective. However, other phenolic starting chemicals, such as phenol and syringol, could be used. Additionally, there is literature showing that increasing the distance between the phenolic units also decreases estrogenic activity of BPA alternatives.

Also, more complex phenolic compounds can be used in the reaction. These molecules include, but are not limited to, cardanol and cardol, compounds that form a significant portion of cashew nut oil, capsaicin, their derivatives and other such compounds.

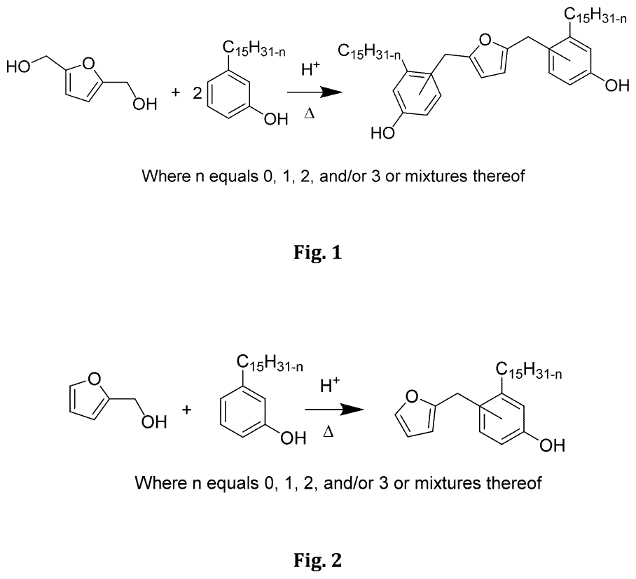

### (FIG. 1)

PFP does not need to be a symmetrical molecule. As a result, multiple phenolic compounds can be mixed to react with the furan to produce the desired product while still achieving the performance and other benefits.

Additionally, furfuryl alcohol can be reacted with phenolics, such as cardanol (FIG. 2) to produce mono-hydroxy containing furan-phenolics. This species can be grafted onto polymer chains or can be used as a reactive diluent in vinyl/(meth)acrylate polymers.

The hydroxyl functional PFP can be modified into epoxy monomers, amines, methacrylates, vinyl esters, polycarbonates, polyamides, polyimides, and polyesters using known chemical procedures described below to show the potential derivatives that can be made from PFP. Since the core molecule, PFP, is novel, these derivative monomers are also novel.

Diglycidyl ethers of substituted bisphenols can be synthesized from PFP to produce Product 1:

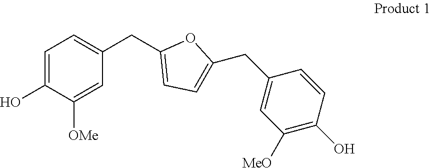 [SMILES: COc1cc(Cc2ccc(Cc3ccc(O)c(OC)c3)o2)ccc1O]

via reaction with epichlorohydrin and a base, which may be an alkali salt, for example sodium hydroxide or potassium hydroxide. The value of n may range from 0 to 24, or from 0 to 10, or from 0 to 5, or from 0 to 3, or from 0 to 1. In some embodiments, synthesis of these diglycidyl ethers is carried out with at least two equivalents of epichlorohydrin, preferably 10 to 30 equivalents, to minimize oligomerization and thereby produce epoxies with average n values less than 1, and with at least two stoichiometric equivalents of base, preferably 3-6 equivalents of base, for every equivalent of substituted bisphenol. The reaction of PFP with epichlorohydrin can be catalyzed by a phase transfer catalyst, which may be a quaternary ammonium salt, for example n-butyl ammonium bromide, preferably at a concentration of 10-11 mol. % of PFP.

The synthesis of the diglycidyl ether of PFP (DGEPFP-Product 2) involves mixing PFP with epichlorohydrin at 15-60° C., preferably 20-25° C., followed by addition of alkali base at 0-10° C., preferably 0-5° C. DGEPFP is recovered from the reaction mixture after aqueous washes to remove salts and distillation to remove epichlorohydrin. The addition of epoxide groups to the substituted bisphenol is confirmed via the presence characteristic epoxide peaks in NMR and near-IR. Epoxide equivalent weight titration as described in ASTM D-1652 is used to determine the average molecular weight per epoxide group.

DGEPFP can be reacted with curing agents such as diamines to create a cross-linked polymer network. Reaction of DGEPFP with a diamine, for example 4,4′-diaminodicyclohexylmethane, preferably at stoichiometric equivalents based on epoxide equivalent weight and amine hydrogen equivalent weight (52.5 g/eq if 4,4′-diaminodicyclohexylmethane) can be carried out at 100-250° C., preferably 160-180° C., with a step curing procedure. The extent of cure is determined via the ratio of epoxy and amine peaks in Near-IR spectra both before and after curing. The glass transition temperature (Tg) of the epoxy resin can be determined via DSC. These diepoxies can also be cured with acid anhydrides in stoichiometric equivalents, thereby creating ester linkages.

More generally, the reaction used to form the epoxy thermoset also involves at least one epoxy curing agent. Suitable curing agents for epoxies are well known in the industry. Examples include aliphatic polyamines such as diethylenetriamine (DETA), triethylenetetramine (TETA), tetraethylenepentamine (TEPA), diproprenediamine (DPDA), dimethylaminopropylamine (DEAPA); alicyclic polyamines such as N-aminoethylpiperazine (N-AEP), menthane diamine (MDA), isophoronediamine (IPDA); aliphatic aromatic amines such as m-xylenediamine (m-XDA); aromatic amines such as metaphenylene diamine (MPDA), diaminodiphenylmethane (DDM), diaminodiphenylsulfone (DDS); and mixtures thereof. Further examples of suitable curing agent include EPIKURE® Curing Agent W, and AMICURE® PACM/bis-(p-aminocyclohexyl) methane. Other curing agents include nadic methyl anhydride, phthalic anhydride dicyandiamide, nadic anhydride, and dicyandiamide. These curing agents are added to epoxy resins in amounts typically at or near stoichiometry, although off-stoichiometry amounts may be useful for the creation of prepregs. Epoxy homopolymerization catalysts, for example tertiary amines such as such as benzyl dimethylamine, can also cure these epoxy resins when added in catalytic amounts, typically up to 5 wt. %. All of the epoxy resins may be cured by ambient, thermal, induction, electron beam, UV cure or other such standard methods whereby energy is provided to initiate the reaction between the epoxy and the curing agent/catalyst. Post-cure is typically necessary because the rate of cure slows severely upon vitrification.

PFP can be functionalized through a number of methods and converted to Product 3 or Product 4 to produce methacrylated and acrylated phenolics, respectively, that are capable of free radical polymerization. Product 3 is formed by esterification of Product 1 using either methacryloyl chloride or methacrylic anhydride and a base catalyst (for example 4-(dimethylamino)pyridine and triethylamine) in an aprotic solvent (for example dichloromethane, and tetrahydrofuran). Reaction preferably occurs at 20-80° C., but most preferably at 25-55° C.

The synthesis of Product 4 can be carried out using a similar methodology employing acryloyl chloride or acrylic anhydride as the (trans) esterification agents. NMR analysis shows peaks in the expected locations, with minimal impurities. Product 2 can be converted to Product 5 by reaction with a slight excess of acrylic acid or to Product 6 using methacrylic acid at 70-120° C., and preferably at 90-100° C., preferably using a catalyst, such as AMC-2 or triphenylphosphine, triphenylantimony (III), for 1-5 hours and preferably 2-3 hours with no separation. Acid number can be used to verify addition of the (meth)acrylic acid with an acid number of less than 20 being ideal. NMR can be used to verify that nearly two (meth)acrylates per molecule are present.

Product 2 can be converted to an epoxy-(meth)acrylic ester by reaction with acrylic acid or methacrylic acid at 70-120° C., preferably 90-100° C., using a catalyst, such as AMC-2 or triphenylphosphine, triphenylantimony (III), for 1-5 hours and preferably 2-3 hours with no separation. The amount of (meth)acrylic acid used is less than the stoichiometric amount of epoxy on Product 2, preferably 25-75 mol. % of the stoichiometric amount. Acid number can be used to verify addition of the (meth)acrylic acid, with an acid number of less than 15 being ideal. NMR can be used to verify the number of (meth)acrylates and epoxies per molecule present.

Product 7 can be synthesized under various conditions that can result in the formation of polyester or unsaturated polyester resins (UPEs) depending on the reaction composition. Product 1 is melted together in the presence or absence of another diol or polyol moiety, for example, diethylene glycol, isosorbide or propylene glycol, with a single organic diacid or a mixture of organic diacids, for example maleic anhydride, phthalic anhydride, terephthalic acid or adipic acid. The reaction is catalyzed using an acid catalyst, for example p-toluenesulfonic acid, AMBERLYST 15 hydrogen form or DOWEX DR-2030 hydrogen form, and can be done in the presence or absence of an azeotropic solvent, for example toluene and xylenes, to aid in water removal. The reaction can be carried out at preferably 55-220° C., but most preferably 125-180° C. NMR analysis showed peaks in the expected locations for polymeric material, based on the components in the starting reaction mixture. GPC analysis showed that the preferred molecular weights are greater than 2,000 g/mol, but molecular weights above 500 g/mol are acceptable, and the most preferred molecular weights of 1,500-3,000 g/mol are also possible.

Product 8 can be synthesized using Product 1 in combination with various diisocyanates or polyisocyanates to form prepolymeric oligomers or high molecular weight polymers, depending on stoichiometric ratios. Product 1 is dissolved in solvent, for example tetrahydrofuran, chloroform and/or diethyl ether, with a multifunctional isocyanate, for example toluene diisocyanate, hexamethylene diisocyanate, methylene diphenyl diisocyanate, and/or isophorone diisocyanate, before adding a catalytic amount of organic base, for example triethylamine, pyridine, or 1,8-diazabicyclo[5.4.0]undec-7-ene (DBU), typically at a concentration of 1-25 mol. %, more typically 5-15 mol. %. The preferred ratios for the synthesis of Product 8 are 25-75 mol. % Product 1 and 25-75 mol. % diisocyanate, more preferably a ratio of 33-67 mol. % Product 1 and 33-67 mol. % diisocyanate. The reaction temperature is preferably 0-125° C., and more preferably 25-80° C. NMR analysis showed peaks in the expected locations for polymeric material without degradation of the starting BGF ring system. GPC analysis showed that the preferred molecular weights are greater than 8,000 g/mol, but weights of 1,500-9,000 g/mol are also possible and the reaction can be completed so that the molecular weights are >12,000 g/mol.

Product 9 can be synthesized using Product 1 in the presence of phosgene or phosgene derivatives or in the presence of p-nitrophenyl chloroformate or other chloroformates. Product 1 can be dissolved in a solvent, for example 1,4-dioxane, acetonitrile and/or dichloromethane. In the case of liquid of solid co-reactants, the co-reactant can be dissolved in a solvent, for example 1,4-dioxane, acetonitrile, dichloromethane. These solutions can be added to a catalytic amount of organic base including, but not limited to pyridine, 4-(dimethylamino)pyridine, 1-methylimidazole and 2-methylimidazole, in concentrations of preferably 0.5-10 mol. %, but most preferably 1-5 mol. %. A stoichiometric amount of a second organic base, for example trimethylamine or pyridine, can also be added. Preferred reaction temperatures are 0-100° C., and more preferably 15-40° C. The reaction may be conducted in contact with atmospheric air, but is preferably carried out under an inert atmosphere. Polymeric material can be recovered by addition of an anti-solvent, but other techniques are possible including filtration, vacuum distillation, chromatography, and flash chromatography. GPC, FTIR and NMR analyses showed peaks in the expected locations for polymeric material without degradation of the starting bisphenolic structure. These results validated that the polymerization is insensitive to the specific structure of Product 1 and thus would be expected to work for any variants of Product 1.

Higher molecular weight polymers can be achieved via higher purity reagents, and optimized reaction conditions. Preferred number average molecular weights (measured by GPC) are greater than 6,000 g/mol, but number average molecular weights of 500-12,000 g/mol are also possible and the reaction can be completed so that the number average molecular weights are greater than 12,000 g/mol. A dispersity of 1-5 is preferred, more preferably 1.5-2.5. In one example, a Tg of 110° C. was determined via DSC (10° C./min heating rate). Typically, the glass transition temperature will be in the range of 25-150° C., more typically 75-150° C.

Product 10 can be prepared using the Smiles re-arrangement or other techniques to convert the hydroxyl group to an amine. PFP (4.4 mmol), with excess 2-chloroacetamide (10.5 mmol), potassium carbonate (3.03 g, 21.9 mmol, 500 mol % BPA), and potassium iodide (0.291 g, 0.9 mmol, 40 mol % BPA) were charged to a round-bottom flask equipped with magnetic stir bar. DMF (20 mL) was added as the reaction solvent. The reaction was conducted at 90° C. for one hour followed by 150° C. for four hours. The reaction mixture was filtered to remove catalyst and then concentrated under reduced pressure. The concentrated reaction mixture was then purified using flash chromatography using a solvent gradient of 54% ethyl acetate in hexanes for 4 min, increasing to 100% ethyl acetate over 14 min. The fractions were then concentrated under reduced pressure.

Product 10 can be cured with Product 2 or other epoxies using methods for curing high temperature epoxy resins. Product 10 can also be cured with esters or anhydrides to yield polyamides and polyimides. Anhydrides such as nadic anhydride (NA) and 3,3′,4,4′-benzophenonetetracarboxylic dianhydride (BTDA) can be reacted with Product 10 to yield a polyamide and a polyimide. The anhydrides were charged to a reactor in pellet form and 100 mL of methanol was added for 30.44 g of these two anhydrides. The anhydrides we are heated in the methanol for about 90 min at 90° C., allowing them time to esterify. After heating for 90 min, the mixture was cooled to room temperature and crushed Product 10 was slowly added. The mixture was then stirred overnight. The ratio of the anhydride functionality to the amine functionality and the ratio of the anhydrides to each other controls the molecular weight of the products. If thermoplastic polyimides are desired, no nadic anhydride should be used and the ratio of BTDA and diamine should be approximately 1:1. For PMR type polyimides, the standard ratio of 2:2.087:3.087 for NA:BTDA:diamine would mimic that which is used for making PMR-15. The oligomers would then be cured under high heat (250° C.) for a few hours to produce a crosslinked thermoset.

Any of the phenolic blocks and/or reactive functionalized phenolics, prepared and cured as discussed above with respect to the exemplary embodiments in Scheme 1, may be used to prepare thermosetting compositions, examples of which include coatings and composite materials.

Coatings made from the cured phenolic blocks and/or reactive functionalized phenolics may contain solvents, for example methyl ethyl ketone, acetone, tert-butyl acetate. The coatings may also contain additional additives such as fibers, clays, silicates, fillers, whiskers or other conventional filler or reinforcing materials, including the nanometer scale analogues thereof; pigments such as titanium dioxide, iron oxides, and carbon black; and corrosion inhibitors such as zinc phosphate. Additional additives that may be employed include flow additives, film formers, defoamers, coupling agents, antioxidants, stabilizers, flame retardants, reheating aids, plasticizers, flexibilizers, anti-fogging agents, nucleating agents, and combinations thereof.

The coatings can be applied using various methods, for example using a brush, roller, or sprayer. The coatings are typically cured under ambient conditions, but may be cured under a variety of other conditions, for example oven curing at elevated temperature. The phenolic blocks and/or reactive functionalized phenolics may be cured by any of the methods and chemistries described herein.

Composites made from the cured phenolic blocks and/or reactive functionalized phenolics may contain additives such as fibers, clays, silicates, fillers, whiskers or other conventional filler or reinforcing materials, including nanomaterials. Typical fibers used for such composites include, but are not limited to, E-glass, S-glass, KEVLAR®, carbon fiber, and ultra-high molecular weight polyethylene. Additional additives may be employed in conventional amounts and may be added directly to the process during formation of the composite. Such additional additives may include, for example, colorants, pigments, carbon black, chopped fibers or particulates of glass, carbon and aramid, fillers, impact modifiers, antioxidants, stabilizers, flame retardants, reheating aids, crystallization aids, oxygen scavengers, plasticizers, flexibilizers, anti-fogging agents, nucleating agents, foaming agents, mold release agents, and combinations thereof. The phenolic blocks and/or reactive functionalized phenolics may be cured by any of the methods and chemistries described herein.

The following exemplary embodiments relate to the specific compounds shown in Scheme 1, but the methods described below can be applied to all embodiments of the invention.

The neat (meth)acrylic ester products (Products 3, 4, 5, & 6) can be treated with a free-radical initiator (for example cumene hydroperoxide and methyl ethyl ketone peroxide) at a concentration of preferably 0.5-8.0 wt. % and most preferably 1.0-3.0 wt. % in order to induce curing of the resin to form a novel polymer. Curing of the resins can be accomplished with or without a promoter, for example cobalt naphthenate and dimethyl aniline, to accelerate gel time, preferably in concentrations of 0.10-1.5 wt. %, and most preferably 0.25-0.5 wt. %. Cure temperatures for substituted bisphenol resins can range from 20-85° C., or preferably at 25-60° C. and preferably the polymers are post-cured at 100-250° C., most preferably at 120-180° C. The novel materials have properties comparable to commercial polymers derived from (meth)acrylic esters and exhibit similar stiffness, toughness and Tg.

The substituted bisphenol (meth)acrylated products (Products 3, 4, 5, & 6) can be blended with one or more reactive diluents, including, but not limited to, styrene, methacrylated lauric acid, and furfuryl methacrylate, to produce novel resin systems. Typically, such compositions contain 30-90 wt. % substituted bisphenol (meth)acrylic ester and 10-70 wt. % reactive diluent, more preferably 50-75 wt. % substituted bisphenol (meth)acrylic ester and 25-50 wt. % reactive diluent. These resins have very low viscosities that would make them ideal for liquid molding, composite layups and vacuum assisted resin transfer molding (VARTM), as well as for a wide range of other applications. These resins can be cured using a free-radical initiator, in the presence or absence of a promoter, to produce BGF co-polymers that have properties similar to polymeric materials produced by existing commercial processes, providing equivalent stiffness, toughness and Tg. The polymer produced from BGF dimethacrylate blended with 50 wt. % styrene was found to have a Tg of 186° C. by DSC at 10° C./min, and a maximum degradation temperature of 380° C. by TGA in nitrogen at 10° C./min.

Substituted bisphenol UPE (Product 7) resin systems can be blended with olefinically unsaturated reactive diluents, including, but not limited to, styrene, methacrylated lauric acid, and methyl methacrylate, to produce novel resin systems where the composition is 30-90 wt. % Product 7 and 10-70 wt. % reactive diluent, preferably 50-75 wt. % Product 7 and 25-50 wt. % reactive diluent. These resins have demonstrated viscosities that are amenable to liquid molding, composite layups, and VARTM processing as well as a wide range of other applications. The blended Product 7 resin can be treated with a free-radical initiator, for example cumene hydroperoxide and methyl ethyl ketone peroxide, at a concentration of 0.5-8.0 wt. %, preferably 1.0-3.0 wt. %, in order to induce curing of the resin to form a novel thermoset polymer. Curing of the resins can be accomplished with or without a promoter, for example cobalt naphthenate and dimethyl aniline, to accelerate gel time preferably in concentrations of 0.10-1.5 wt. %, and more preferably 0.25-0.75 wt. %. Cure temperatures for these UPE resins can range from 20-85° C., preferably 25-60° C. and the polymers can be post-cured at 100-200° C., preferably at 120-180° C.

Alternatively, high molecular weight polyester polymers can be prepared and used as is, in applications such as clothing and beverage bottles. In this case, the stoichiometry of the PFP and a carboxylic acid or acid chloride must be nearly 1, e.g. 0.8-1.2, preferably 0.9-1.1 or, most preferably 0.95-1.05, to enable high degrees of polymerization.

NMR results confirm the preparation of the following compounds that demonstrate that a variety of PFP compounds can be made, and also demonstrates that a variety of PFP derivatives can also be made. The preparation procedures for the derivatives that were made are sufficient to demonstrate that the procedures for making the derivatives are generally applicable. Additionally, many different varieties of PFP compounds can be prepared using this invention by, for example, the use of different starting chemicals, including compounds such as syringol. Exemplary PFP compounds include:
1) Guaiacol-furan-guaiacol
2) Diglycidyl ether of Guaiacol-furan-guaiacol
3) Dimethacrylate of Guaiacol-furan-guaiacol
4) m-cresol-furan-m-cresol
5) o-cresol-furan-o-cresol
6) diglycidyl ether of o-cresol-furan-o-cresol
7) dimethacrylate of o-cresol-furan-o-cresol
8) Phenol-furan-phenol

The advantages of this PFP resin system over BPA/F are as follows:
1) Reduced toxicity
   a. PFP with methoxy functional groups on the phenolic groups should reduce the toxicity of the molecule relative to BPA.
   b. The longer spacer between the phenolic units should further reduce toxicity.
2) Reduced fluid permeation: Furan groups increase the density of the polymer and thereby decrease gas and water permeability through the polymer. This could be useful for corrosion resistance, food packaging, and other applications. The long fatty acid chain on cardanol enables reduced water solubility and permeability.
3) Improved polymer properties: PFP resins may have improved thermal properties, in particular char content, increased toughness, and increased glassy modulus.
4) Self-healing capability due to the furan component. These molecules can provide self-healing capability because a dienophile, such as bismaleimide, can be dispersed throughout the polymer, added in microcapsules or grafted to the polymer to react with the furan diene. The resulting Diels-Alder reaction will create chemical linkages that can self-heal cracks or other damage done to the polymer.
5) Anti-fouling benefits: Capsaicin is the active component of chili peppers. Use of capsaicin enables formation of anti-fouling, anti-fungal, etc. products because bacteria and other organisms generally tend to avoid capsaicin and cannot proliferate when in contact with it.
6) Medicinal benefits: Capsaicin is an anti-inflammatory agent and thus this methodology may enable development of products that exhibit local anti-inflammatory activity, such as for use in polymers and coatings used for implants, bandages, sutures, and other applications. Furthermore, PFP made from capsaicin could be included in oral delivery systems to allow time release of the anti-inflammatory agent, using the capsaicin to not only produce the inflammatory response, but also to produce a polymer that would dissolve over time to provide the time release. Only the surface coating of the material would need to include small amounts of capsaicin in order to provide the anti-inflammatory activity, and thus the relatively low production volumes of capsaicin would not be a major problem for this application.
7) Makes use of renewable chemistry
   a) bHMF is derived from biomass processing. Two major benefits of using bHMF over vanillyl alcohol is that bHMF is likely to be produced at a much higher volume and that bHMF does not compete with food needs.
   b) Cardanol, guaiacol, capsaicin, and other phenolic compounds can be derived from renewable compounds. A major benefit of using cardanol vs phenol or guaiacol is that cardanol is highly renewable and can be produced in significant quantities.
8) The phenolic component, regardless of which phenolic monomer is chosen, can be derived from biomass and/or petroleum to balance production requirements and environmental sustainability.

The invention was designed to reduce the toxicity of BPA/F without having to use vanillin. Vanillin is a relatively expensive component while bHMF is a by-product produced during the conversion of biomass to ethanol. Additionally, this invention was designed to produce high performance polymers with unique properties from renewable sources.

The products of the present invention can be used in any application where BPA/F are currently used including epoxy and vinyl ester composites, polycarbonate headlights, epoxy resins for food packaging, epoxy resins for coatings, and methacrylate adhesives for dental and structural applications. Also, the products of the present invention can be used for anti-fouling coatings, anti-inflammatory medicines and coatings.

What is claimed is:

1. A furan containing compound according to Formula (I), 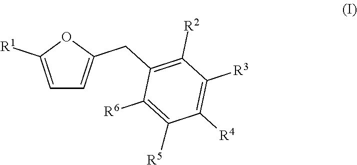
wherein R¹ is 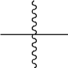
wherein 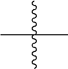
indicates a bond that is a point of attachment to a group according to Formula (II): 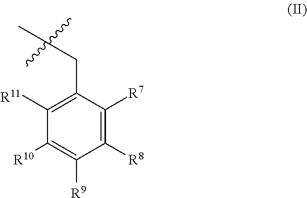
wherein R², R³, R⁴, R⁵, R⁶, R⁷, R⁸, R⁹, R¹⁰, and R¹¹ are each independently selected from: hydrogen, halogen, hydroxy, amino, nitro, cyano, carboxy, alkylamine residues having 1 to 18 carbon atoms, aminoalkyl residues having 1 to 18 carbon atoms, alkenylamine residues having 1 to 18 carbon atoms, aminoalkenyl residues having 1 to 18 carbon atoms, alkylamide residues having 1 to 18 carbon atoms, amidoalkyl residues having 1 to 18 carbon atoms, alkenylamide residues having 1 to 18 carbon atoms, amidoalkenyl residues having 1 to 18 carbon atoms, an optionally substituted alkyl group having 1 to 20 carbon atoms, an optionally substituted alkenyl group having 2 to 20 carbon atoms, an optionally substituted alkoxy group having 1 to 20 carbon atoms, an optionally substituted cycloalkyl group having 3 to 12 carbon atoms, an optionally substituted aryl group having 6 to 16 carbon atoms, and an optionally substituted heterocyclic group having 3 to 16 carbon atoms; wherein the alkyl group, the alkenyl group, the alkoxy group, the cycloalkyl group, the aryl group and the heterocyclic group can be substituted with 1 to 5 substituents independently selected from halogen, hydroxy, amino, nitro, cyano, carboxy, an alkyl group having 1 to 20 carbons, a heterocyclic group having 3 to 16 carbons, and an alkoxy group having 1 to 20 carbon atoms; wherein one or more of R²-R⁶ is hydrogen and one or more of R²-R⁶ is a hydroxy or amino; and wherein one or more of R⁷-R¹¹ is a hydroxy or amino, with the proviso that when R¹ is —H, R² and R⁵ cannot both be —OH at the same time that all of R³, R⁴ and R⁶ are hydrogen.

2. The compound of claim 1, wherein the furan containing compound is a compound of Formula (III): 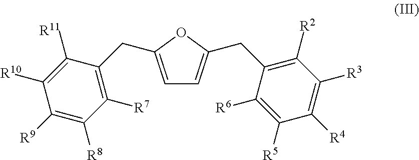
wherein R⁴ and R⁹ are each independently selected from hydroxy or amino groups.

3. A compound which is a reaction product prepared by the reaction of:
i) the compound of Formula (I) wherein R¹ is 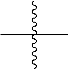
as recited in claim 1; and ii) a reagent selected from one of the following:
a) a radically polymerizable monomer;
b) a halo-containing epoxide;
c) at least one diacid, anhydride or diacyl chloride;
d) an isocyanate selected from hexamethylene diisocyanate, isophorone diisocyanate, and methylenediphenyl diisocyanate;
e) at least one compound selected from phosgene, diphosgene, triphosgene, and p-nitrophenyl chloroformate; and
f) a compound for converting a hydroxy to at least one of an amine and amide, and wherein at least one of R²-R⁶ is a hydroxy and at least one of R⁷-R¹¹ is a hydroxy.

4. The compound of claim 3, wherein the reaction product is formed from the radically polymerizable monomer reagent, and the radically polymerizable monomer reagent is selected from methacryloyl chloride, methacrylic anhydride, acryloyl chloride, acrylic anhydride, acrylic acid, and methacrylic acid, and wherein in the reaction product, a carbonyl of the radically polymerizable monomer is bonded to the oxygen from the hydroxy.

5. The compound of claim 3, wherein the reaction product is formed from the radically polymerizable monomer reagent, the radically polymerizable monomer reagent is selected from methacryloyl chloride, methacrylic anhydride, methyl methacrylate, and methacrylic acid and the reaction product is a product of Formula (IV):

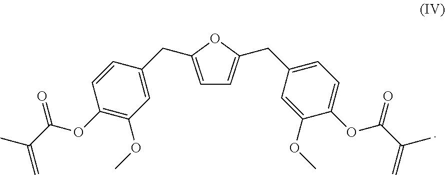 [SMILES: C=C(C)C(=O)Oc1ccc(Cc2ccc(Cc3ccc(OC(=O)C(=C)C)c(OC)c3)o2)cc1OC]

6. The compound of claim 3, wherein the reaction product is formed from the radically polymerizable monomer reagent, the radically polymerizable monomer reagent is selected from acryloyl chloride and acrylic anhydride and the reaction product is a product of Formula (V):

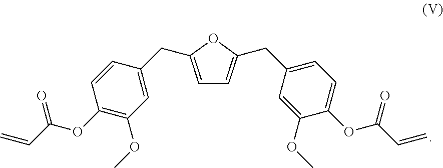 [SMILES: C=CC(=O)Oc1ccc(Cc2ccc(Cc3ccc(OC(=O)C=C)c(OC)c3)o2)cc1OC]

7. A polymer produced by radical polymerization of the reaction product of the compound of claim 4 formed by reaction with the radically polymerizable monomer reagent.

8. A polymer produced by further reacting the reaction product of claim 4 formed with the radically polymerizable monomer reagent, with a reactive diluent selected from styrene, methacrylated lauric acid, and furfuryl methacrylate and wherein 30-90 wt. % of the reaction product formed with the radically polymerizable monomer reagent is reacted with 10-70 wt. % of the reactive diluent.

9. The compound of claim 3, wherein the reaction product is formed from the compound of Formula (I) and the halo-containing epoxide.

10. The compound of claim 9, wherein the reaction between the compound of Formula (I) and the reagent which is the halo-containing epoxide is is performed in the presence of a base and is catalyzed by a phase transfer catalyst.

11. The compound of claim 3, wherein at least one of R²-R⁶ is a hydroxy and at least one of R⁷-R¹¹ is a hydroxy, the reaction product is formed from the halo-containing epoxide, and the reaction product is substituted on the oxygen of one said hydroxy group of R²-R⁶ and on the oxygen of one said hydroxy group of R⁷-R¹¹ with alkyl epoxy groups.

12. The compound of claim 11, wherein the reaction product is:

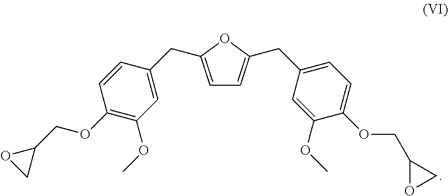 [SMILES: COc1cc(Cc2ccc(Cc3ccc(OCC4CO4)c(OC)c3)o2)ccc1OCC1CO1]

13. An epoxy thermoset formed by curing, in the presence of at least one epoxy curing agent, the reaction product of claim 3 formed from the compound of Formula (I) and the halo-containing epoxide.

14. The epoxy thermoset of claim 13, wherein the epoxy curing agent is an aliphatic polyamine, which is preferably diethylenetriamine (DETA), triethylenetetramine (TETA), tetraethylenepentamine (TEPA), diproprenediamine (DPDA), or dimethylaminopropylamine (DEAPA); or an alicyclic polyamine which is preferably N-aminoethylpiperazine (N-AEP), 4,4′-diaminodicyclohexylmethane (AMICURE® PACM), menthane diamine (MDA), or isophoronediamine (IPDA); or an aliphatic aromatic amine which is preferably m-xylenediamine (m-XDA); or an aromatic amine which is preferably metaphenylene diamine (MPDA), diaminodiphenylmethane (DDM), diaminodiphenylsulfone (DDS); or EPIKURE® Curing Agent W; or nadic methyl anhydride, phthalic anhydride dicyandiamide, nadic anhydride, and dicyandiamide, hexahydrophthalic anhydride (HHPA), methylhexahydrophthalic anhydride (MHHPA) and methyltetrahydrophthalic anhydride (MTHPA).

15. A polymerizable reaction product of the compound of claim 9 and a radically polymerizable monomer selected from acrylic acid and methacrylic acid.

16. The polymerizable reaction product of claim 15, wherein a molar ratio of the radically polymerizable monomer to the compound of claim 9 is from 1:1 to 2:1, preferably from 1.1:1 to 1.5:1.

17. The compound of claim 15, wherein the polymerizable reaction product further comprises a reactive diluent, and said reactive diluent is preferably styrene, methacrylated lauric acid, or furfuryl methacrylate.

18. The compound of claim 15, wherein the radically polymerizable monomer is acrylic acid and forms a reaction product according to Formula (VII):

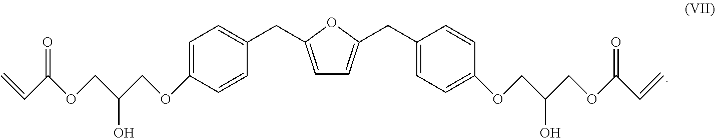 [SMILES: C=CC(=O)OCC(O)COc1ccc(Cc2ccc(Cc3ccc(OCC(O)COC(=O)C=C)cc3)o2)cc1]

19. The compound of claim 15, wherein the radically polymerizable monomer is methacrylic acid and forms a reaction product according to Formula (VIII):

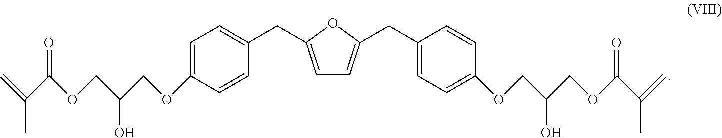 [SMILES: C=C(C)C(=O)OCC(O)COc1ccc(Cc2ccc(Cc3ccc(OCC(O)COC(=O)C(=C)C)cc3)o2)cc1]

20. The compound of claim 3, wherein the reagent is selected from at least one diacid, at least one anhydride, at least one diacyl chloride and mixtures thereof.
